(19) United States
(12) Patent Application Publication
(10) Pub. No.: US 2021/0275543 A1
(43) Pub. Date: Sep. 9, 2021

(54) METHODS FOR THE TREATMENT OF MITOCHONDRIAL GENETIC DISEASES

(71) Applicants: NSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); UNIVERSITÉ PARIS DESCARTES, Paris (FR); ASSISTANCE PUBLIQUE-HÔPITAUX DE PARIS (APHP), Paris (FR)

(72) Inventor: Guillaume CANAUD, Paris (FR)

(21) Appl. No.: 16/497,704

(22) PCT Filed: Mar. 29, 2018
(86) PCT No.: PCT/EP2018/058075

§ 371 Date: Sep. 25, 2019

(30) Foreign Application Priority Data
Mar. 30, 2017 (EP) 17305373.7

(51) Int. Cl. A61K 31/553; A61K 31/4439; A61P 25/28; C12Q 1/48

(52) U.S. Cl. CPC A61K 31/553; A61K 31/4439; G01N 2333/91215; C12Q 1/485; A61P 25/28

(57) ABSTRACT

The invention relates to a method for treating mitochondrial genetic diseases. The inventors have worked with primary fibroblasts from patients and control individuals and collected protein lysates for western blotting. Importantly, they observed that the genetic mitochondrial disorders, show a significant increase in phosphorylation of ribosomal protein S6 (pS6) compared to control fibroblasts, indicative of hyperactivated mTOR signaling. Patients with mitochondrial disorders and controls cells were treated for 48 hours with DMSO or BYL719. All lines from patients with mitochondrial diseases show reduced membrane potential, determined by TMRE staining intensity, and abnormal morphology, fragmentation and the presence of depolarized (low TMRE staining) mitochondria. Treatment with BYL719 attenuated these phenotypes in all MELAS fibroblasts while having no overt impact on the control cells. Similar experiments using flow cytometry confirmed membrane potential (TMRE) rescue by BYL719 treatment in MELAS fibroblasts.

### FIELD OF THE INVENTION

The invention relates to method and compositions for the treatment of mitochondrial genetic diseases, such as mitochondrial cytopathy.

### BACKGROUND OF THE INVENTION

Mitochondrial diseases are a clinically and genetically heterogeneous group of disorders that arise as a result of mitochondrial dysfunction¹. Inherited mitochondrial diseases can be caused by mutations of mitochondrial DNA (mtDNA) or of nuclear genes that encode mitochondrial proteins, with an overall prevalence estimated at approximately 1 in 5,000¹. The clinical phenotypes of patients affected by mitochondrial disorders are considerably heterogeneous². Individuals with mitochondrial disorders resulting from mutation of mtDNA may harbor a mixture of mutated and wild-type mtDNA within each cell. The percentage of mutant mtDNA varies between individuals, as well as among organs and tissues within the same individual, contributing to the varied clinical phenotype³.

Currently, there is no effective treatment for mitochondrial diseases of any etiology and management is mainly supportive. In small uncontrolled studies, vitamins C and K, thiamine, riboflavin, and ubiquinone have shown varying degrees of benefit in individual cases⁴. Recent experimental studies have demonstrated that reduced mTOR signaling through caloric restriction or genetic manipulation rescues the lifespan of yeast harboring mutations in genes encoding mitochondrial proteins′. Furthermore, we recently reported a robust attenuation of disease in a mouse model of the human mitochondrial disease Leigh syndrome (Ndufs4−/− mice) using pharmacological inhibition of mTOR6⁷. We observed that the Ndufs4−/− mice show increased tissue mTOR activation in affected tissue (brain) associated with metabolic defects and progressive neurological disease. Importantly, we found that rapamycin, a specific inhibitor of mTOR, substantially delayed the onset of neurological symptoms and lesions and extended the lifespan of the Ndufs4−/− mice⁶.

Rapamycin is an immunosuppressive drug used after solid organ transplantation to prevent allograft rejection^{8,9} but is frequently associated with severe side effects that limit its use¹⁰. Other therapeutics any indeed mandatory. Thus, there is a need to find new therapeutic strategy to treat mitochondrial genetic diseases.

### SUMMARY OF THE INVENTION

The present invention relates to a method for treating mitochondrial genetic diseases in a subject in need thereof comprising a step of administrating the subject with a therapeutically effective amount of PI3K inhibitor. In particular, the present invention is defined by the claims.

### DETAILED DESCRIPTION OF THE INVENTION

Inventors have worked with primary fibroblasts from patients and control individuals and collected protein lysates for western blotting. Importantly, they observed that the genetic mitochondrial disorders, show a significant increase in phosphorylation of ribosomal protein S6 (pS6) compared to control fibroblasts, indicative of hyperactivated mTOR signaling. Patients with mitochondrial disorders and controls cells were treated for 48 hours with DMSO or BYL719. BYL719 synthesized by Novartis is in clinical trial at phase II/III for advanced solid tumours. All lines from patients with mitochondrial diseases show reduced membrane potential, determined by TMRE staining intensity, and abnormal morphology, fragmentation and the presence of depolarized (low TMRE staining) mitochondria. Treatment with BYL719 attenuated these phenotypes in all MELAS fibroblasts while having no overt impact on the control cells. Similar experiments using flow cytometry confirmed membrane potential (TMRE) rescue by BYL719 treatment in MELAS fibroblasts.

Accordingly, the present invention relates to a method for treating mitochondrial genetic diseases in a subject in need thereof comprising a step of administrating the subject with a therapeutically effective amount of PI3K inhibitor.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the term “mitochondrial genetic diseases” refers to a clinically and genetically heterogeneous group of disorders that arise as a result of mitochondrial dysfunction^{1.} Mitochondrial genetic disorders are caused by mutations in either the mitochondrial DNA or nuclear DNA that lead to dysfunction of the mitochondria and inadequate production of energy. Example of mitochondrial genetic disorders: mitochondrial cytopathy, aminoglycoside induced deafness; chronic progressive external ophthalmoplegia; depletion syndromes; Kearns-Sayre syndrome; Leber's hereditary optic neuropathy; Leigh syndrome; Cerebellar Hypoplasia, Mitochondrial myopathy Encephalopathy Lactic Acidosis, and Stroke-like episodes (MELAS); myoclonic epilepsy and ragged red fibres; maternally inherited Leigh syndrome; neurogenic weakness, ataxia, and retinitis pigmentosa; Pearson syndrome; microcephaly, optic atrophy, lactic acidosis, Optic nerve atrophy, Spastic paraplegia, Friedreich's ataxia, Sideroblastic anaemia and ataxia, Sideroblastic anaemia, Encephalomyopathy, tubulopathy, ataxia, Hypertrophic cardiomyopathy LS or Alpers syndrome.

As used herein, the term “subject” refers to any mammals, such as a rodent, a feline, a canine, and a primate. Particularly, in the present invention, the subject is a human afflicted with or susceptible to be afflicted with at least one of disorder mitochondrial genetic diseases as described above.

As used herein, the term “PI3K refers to phosphoinositide 3-kinases also called phophatidylinositide 3-kinases. PI3K belongs to a family of enzymes which phosphorylate the 3′hydroxyl group of the onositol ring of the phosphatidylinositol (PtdIns). The PI3K signalling pathway can be activated, resulting in the synthesis of PIP3 from PIP2.

As used herein, the term “PI3K inhibitor” refers to a natural or synthetic compound that has a biological effect to inhibit the activity or the expression of PI3K. More particularly, such compound is capable of inhibiting the kinase activity of at least one member of PI3K family, for example, at least a member of Class I PI3K. In particular embodiment, said PI3K inhibitor may be a pan-inhibitor of Class I PI3K (known as p110) or isoform specific of Class I PI3K isoforms (among the four types of isoforms, p110α, p110β, p110γ or p110δ).

In a particular embodiment, the PI3K inhibitor is a peptide, petptidomimetic, small organic molecule, antibody, aptamers, siRNA or antisense oligonucleotide. The term “peptidomimetic” refers to a small protein-like chain designed to mimic a peptide. In a particular embodiment, the inhibitor of PI3K is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the PI3K inhibitor is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In a particular embodiment, the PI3K inhibitor is a small molecule which is an isoform-selective inhibitor of PI3K selected among the following compounds: BYL719 (Alpelisib, Novartis), GDC-0032 (Taselisib, Genentech/Roche), BKM120 (Buparlisib), INK1117 (Millenium), A66 (University of Auckland), GSK260301 (Glaxosmithkline), KIN-193 (Astra-Zeneca), TGX221 (Monash University), TG1202, CAL101 (Idelalisib, Gilead Sciences), GS-9820 (Gilead Sciences), AMG319 (Amgen), IC87114 (Icos Corporation), BAY80-6946 (Copanlisib, Bayer Healthcare), GDC0941 (Pictlisib, Genentech), IPI145 (Duvelisib, Infinity), SAR405 (Sanofi), PX-866 (Oncothyreon) or their pharmaceutically acceptable salts. Such PI3K inhibitors are well-known in the art and described for example in Wang et al Acta Pharmacological Sinica (2015) 36: 1170-1176.

In a particular embodiment, the PI3K inhibitor is BYL719. As used herein, the term “BYL719” is an ATP-competitive oral PI3K inhibitor selective for the p110a isoform that is activated by a mutant PIK3CA gene (Furet P., et al. 2013; Fritsch C., et al 2014). This molecule is also called Alpelisib and has the following formula and structure in the art C₁₉H₂₂F₃N₅O₂S:

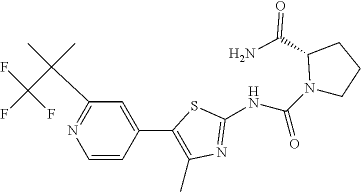 [SMILES: Cc1nc(NC(=O)N2CCC[C@H]2C(N)=O)sc1-c1ccnc(C(C)(C)C(F)(F)F)c1]

In a particular embodiment, the PI3K inhibitor is GDC-0032, developed by Roche. This molecule also called Taselisib has the following formula and structure in the art C₂₄H₂₈N₈O₂:

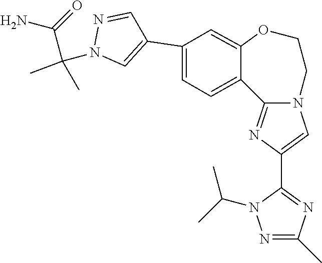 [SMILES: Cc1nc(-c2cn3c(n2)-c2ccc(-c4cnn(C(C)(C)C(N)=O)c4)cc2OCC3)n(C(C)C)n1]

In some embodiments, the PI3K inhibitor is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388. In a particular embodiment, the inhibitor is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the PI3K inhibitor is an intrabody having specificity for PI3K. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In some embodiments, the PI3K inhibitor is a short hairpin RNA (shRNA), a small interfering RNA (siRNA) or an antisense oligonucleotide which inhibits the expression of USP14. In a particular embodiment, the inhibitor of USP14 expression is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of PI3K expression is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339: 823-826), Xenopus tropicalis (Guo et al., 2014, Development, Vol. 141: 707-714.), yeast (DiCarlo et al., 2013, Nucleic Acids Res., Vol. 41: 4336-4343.), Drosophila (Gratz et al., 2014 Genetics, doi:10.1534/genetics.113.160713), monkeys (Niu et al., 2014, Cell, Vol. 156: 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

As used herein the terms “administering” or “administration” refer to the act of injecting or otherwise physically delivering a substance as it exists outside the body (e.g., an inhibitor of PI3K) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The PIK3CA inhibitors as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

A further object of the present invention relates to a method of screening a drug suitable for the treatment of mitochondrial genetic diseases comprising i) providing a test compound and ii) determining the ability of said test compound to inhibit the activity of PI3K.

Any biological assay well known in the art could be suitable for determining the ability of the test compound to inhibit the activity of PI3K. In some embodiments, the assay first comprises determining the ability of the test compound to bind to PI3K. In some embodiments, a population of cells is then contacted and activated so as to determine the ability of the test compound to inhibit the activity of PI3K. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of PI3K, as determined using in vitro methods described herein, are likely to exhibit similar modulatory capacity in applications in vivo. Typically, the test compound is selected from the group consisting of peptides, petptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1: mTOR Activation and Mitochondrial Defects in Primary Fibroblast Lines from patients with mitochondrial disorders. A) Western blotting of lysates from primary MELAS patient and control fibroblast lines showing mTORC1 overactivation in MELAS patients. Cells were treated for 48 hours with DMSO, BYL719, or cyclosporin A (CsA) and probed for phosphorylated ribosomal protein S6, total rpS6, and GAPDH and quantification. 48 hours of treatment with BYL719 inhibited S6 phosphorylation, while treatment with cyclosporin A had no significant impact. B) Western blotting of lysates from other mitochondrial disorders and control fibroblast lines showing mTORC1 overactivation in MELAS patients. Cells were treated for 48 hours with DMSO or BYL719 and probed for phosphorylated ribosomal protein S6, total rpS6, and GAPDH and quantification. 48 hours of treatment with BYL719 inhibited S6 phosphorylation. C) Representative confocal microscopy images of primary fibroblast lines treated for 48 hours with DMSO or BYL719 and stained for mitochondrial membrane potential (tetramethylrhodamine ethyl ester, TMRE, red), mitochondrial mass (10-N-nonyl acridine orange, 10-NAO, green), and DNA (Hoechst 33342, blue).

FIG. 2: daily BYL719 administration dramatically improved animal survival. At the age of 21 days, Ndufs4−/− mice were treated with the PI3KCA inhibitor, BYL719 (MedChem Express; 50 mg·kg-1 in 0.5% carboxymethylcellulose (Sigma Aldrich), daily p.o.) (n=9) or vehicle (0.5% carboxymethylcellulose (Sigma Aldrich), daily p.o.) (n=8).

### EXAMPLES

### Example 1: Rescue of Mitochondrial Morphology and Membrane Potential by Short-Term BYL719 Treatment

Material & Methods

Patients

Nine patients with genetic mitochondrial disorders (MELAS n=4, Leigh Syndrome n=3, Ataxia and Cerebellar Hypoplasia n=1 and Kearns-Sayre Syndrome n=1) and 4 healthy control individuals had a skin biopsy with isolation of dermal fibroblasts. Punch skin biopsies were collected using standard methods.

Generation of Primary Dermal Fibroblast Cultures

To generate dermal fibroblast cultures, biopsies were minced and incubated at room temperature in 0.05% trypsin-EDTA (ThermoFisher 25300054) solution for 30 min with gentle shaking. Cells were collected by centrifuging at 700 g for 10 min, re-suspended in cell culture media containing 25% FBS, and plated onto 24 well plates to establish lines. Fibroblast cultures were grown and maintained in 1×MEM (Corning 10-010-CV) supplemented with 25% FBS and penicillin/streptomycin (Corning 30-001-CI) to a final concentration of 100 IU penicillin and 500 μg/mL streptomycin.

Analysis of Mitochondrial Membrane Potential and Morphology

Cells at similar population doubling (PD-10+/−2) were plated 1:4 from confluent cultures onto coverglass chamberslides and allowed to grow until ˜80% confluent. Media was replaced and supplemented with 5 μmon BYL719 (Chem Express) in DMSO (Fisher BP321-1), or equal volume DMSO, for 48 hours. Cells were stained for 15 min in media with 100 nM tetramethylrhodamine ethyl ester (TMRE, Fisher BDB564696) and 5 μg/mL Hoechst 33342 (Biotium 89139-126) and imaged on a Leica SP5 confocal microscope. Samples were treated and imaged in one session using identical imaging parameters. Flow cytometry analysis was performed by staining cells with only TMRE or 10-N-nonyl acridine orange (10-NAO), dissociating with trypsin-EDTA containing dye for 10 min at room temperature, collecting cells by centrifugation, resuspension in cold PBS, and analysis on a BD Canto II flow cytometer using 488 nm excitation with 585/42 BP and 530/30 BP filters for TMRE and 10-NAO, respectively. A single gate was set to cells using forward and side scatter and all settings unchanged throughout data collection. 10,000 or more events were collected for every sample.

Analysis of S6 Phosphorylation by Western Blotting

Cells at similar population doubling (PD-10+/−2) were plated 1:4 from confluent cultures and allowed to grow until ˜80% confluent. Media was replaced with media containing 5 μmol/L BYL719 (Chem Express) in DMSO or equal volume DMSO for 48 hours. Cultures were rinsed with 1×PBS, treated for 10 min with 0.05% Trypsin, collected by centrifugation at 4° C., and pellets were flash frozen on dry ice. Protein lysates were collected by directly adding 1×RIPA buffer (Pierce 89900) containing protease and phosphatase inhibitors (Pierce PI78441) to cell pellets, sonicating in 10 one-second bursts, on ice, with an XL-2000 QSonica at maximum output, and centrifuging to remove cell debris. Protein concentration was determined by BCA assay (Pierce PI23228), equal protein run on 4-12% bis-tris 26 well NuPage midigels (Fisher WG1403), and transferred to nitrocellulose blots (Fisher IB23001). Blots were blocked in LICOR Odyssey blocking buffer (LICOR 427-40100), probed with primary antibodies anti-pS6, anti-S6, and anti-GAPDH (Cell Signaling 4858P, 2217S, and 2118S, respectively) followed by secondary antibody IRDye800 donkey anti-rabbit (LICOR 925-32213) and imaged using LICOR Odyssey Clx scanning imager as previously described⁹. Data quantified using NIH ImageJ¹¹.

Statistical Analysis

All data were presented as means+/−SEM. Comparisons between groups were performed using student t-tests, 2-tails. P<0.05 was considered significant. Statistical comparisons of capacity curves were performed using the log-rank test as indicated.

Results

Hyperactive mTOR Signaling in Primary Culture of Fibroblasts from Patients with Mitochondrial Disease

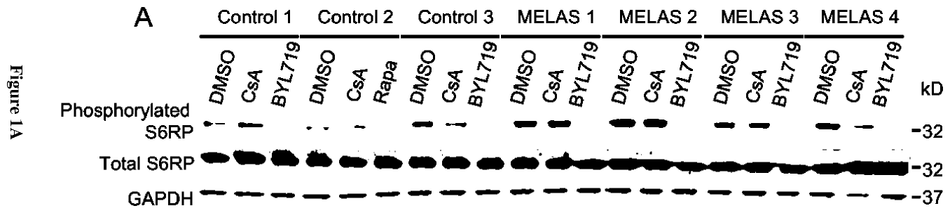

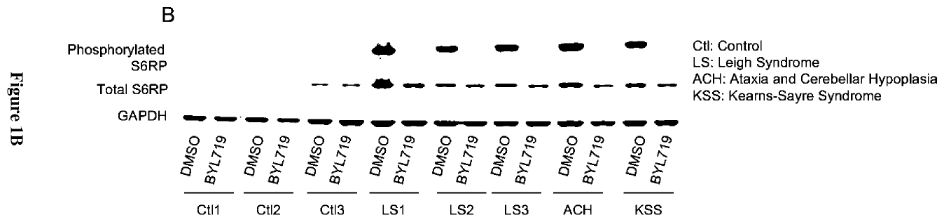

In our previous work we found mTOR to be hyperactivated in whole brain lysates of the Leigh syndrome mouse model⁶. To examine if the mTOR pathway was also hyperactive in genetic mitochondrial disorders, we cultured primary fibroblasts from patients and control individuals and collected protein lysates for western blotting. Importantly, we observed that the genetic mitochondrial disorders, show a significant increase in phosphorylation of ribosomal protein S6 (pS6) compared to control fibroblasts, indicative of hyperactivated mTOR signaling (FIG. 1A). We observed also the same findings in all cell lines with mitochondrial disorders (FIG. 1B).

Patients with mitochondrial disorders and controls cells were then treated for 48 hours with DMSO or BYL719. 48 hours of BYL719 treatment reduced pS6 levels in all cell lines (FIGS. 1A and 1B).

Rescue of Mitochondrial Morphology and Membrane Potential by Short-Term BYL719 Treatment.

To examine the impact of mTOR inhibition on mitochondrial morphology and membrane potential, a general measure of mitochondrial function, we treated dermal fibroblasts with BYL719 or DMSO. Cells were stained with TMRE, a marker of mitochondrial membrane potential, and 10-NAO, a marker of the inner mitochondrial membrane which acts as a membrane potential insensitive marker of mitochondrial mass (FIG. 1C). All lines from patients with mitochondrial diseases show reduced membrane potential, determined by TMRE staining intensity, and abnormal morphology, fragmentation and the presence of depolarized (low TMRE staining) mitochondria. Treatment with BYL719 attenuated these phenotypes in all MELAS fibroblasts while having no overt impact on the control cells (FIG. 1C). Similar experiments using flow cytometry confirmed membrane potential (TMRE) rescue by BYL719 treatment in MELAS fibroblasts (data not shown).

### Example 2: PIK3CA Inhibition in a Mouse Model of Mitochondrial Disorder

Material & Methods

Leigh Syndrome is a severe mitochondrial disease that occurs in about 1:40,000 newborns and is associated with retarded growth, muscular deficits including myopathy and dyspnea, lactic acidosis, and a characteristic progressive necrotizing encephalopathy of the vestibular nuclei, cerebellum, and olfactory bulb (Budde et al., 2002). Ndufs4 encodes a subunit of Complex I of the mitochondrial electron transport chain; mutations in the NDUFS4 gene cause LS in humans (Budde et al., 2000), and the Ndufs4 knockout mouse is a murine model of LS (Kruse et al., 2008). Ndufs4−/− mice have decreased Complex I levels and activity in multiple tissues and show severe and progressive symptoms of mitochondrial disease that mirror human LS. LS results in death at an average of 6-7 years in humans, and Ndufs4 KO mice show a similar early-life mortality with an average lifespan of just 60 days. Heterozygous Ndufs4 knockout mice on a C57Bl/6NIA background were bred to produce homozygous KO animals. Animals were fed ad libitum and housed at a constant ambient temperature in a 12-hour light cycle. Animal procedures were approved by the “Services Vétérinaires de la Prefecture de Police de Paris” Departmental Director and by the ethical committee of the Paris Descartes University.

At the age of 21 days, Ndufs4−/− mice were treated with the PI3KCA inhibitor, BYL719 (MedChem Express; 50 mg·kg-1 in 0.5% carboxymethylcellulose (Sigma Aldrich), daily p.o.) (n=9) or vehicle (0.5% carboxymethylcellulose (Sigma Aldrich), daily p.o.) (n=8).

Results

As previously reported, Ndufs4^{-/-} mice first displayed neurological symptoms with difficulty walking, dyspnea, blindness and finally die around P60.

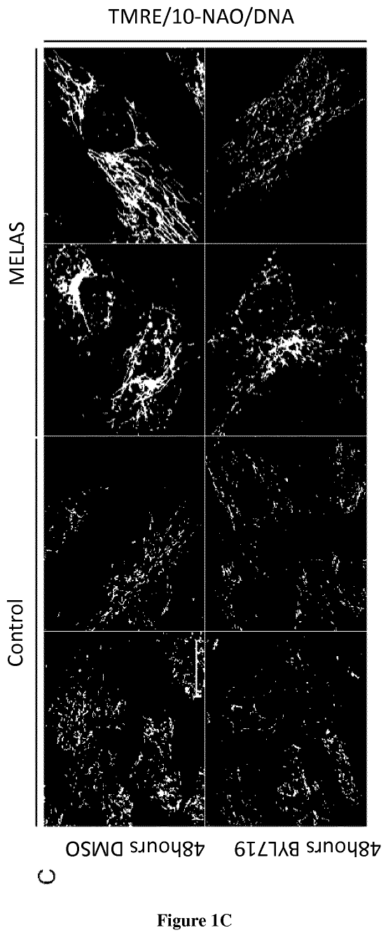

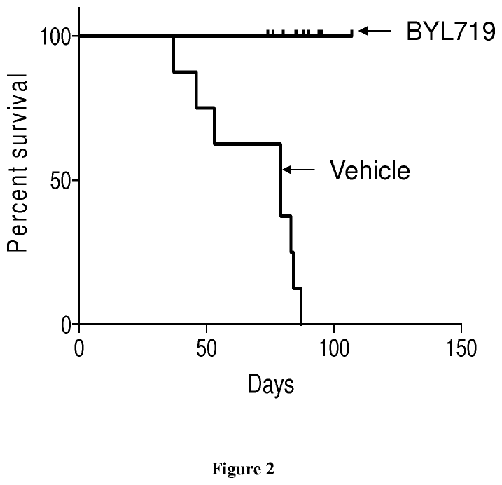

Interestingly, we observed that daily BYL719 administration dramatically improved animal survival (Figure. 2). In fact, while all Ndufs4^{-/-} placebo-treated mice died within 80 days, the BYL719 treated Ndufs4^{-/-} mice were alive 120 days later with an overtly normal appearance.

These results demonstrate the effectiveness of PIK3CA inhibition in a mouse model of mitochondrial disorder.

### REFERENCES

Throughout this application, various references describe the state of the art to which this invention pertains. The disclosures of these references are hereby incorporated by reference into the present disclosure.
1. DiMauro S, Schon E A. Mitochondrial respiratory-chain diseases. N Engl J Med 2003; 348:2656-68.
2. Schon E A, DiMauro S, Hirano M. Human mitochondrial DNA: roles of inherited and somatic mutations. Nat Rev Genet 2012; 13:878-90.
3. Macmillan C, Lach B, Shoubridge E A. Variable distribution of mutant mitochondrial DNAs (tRNA(Leu[3243])) in tissues of symptomatic relatives with MELAS: the role of mitotic segregation. Neurology 1993; 43:1586-90.
4. Dimauro S, Mancuso M, Naini A. Mitochondrial encephalomyopathies: therapeutic approach. Ann N Y Acad Sci 2004; 1011:232-45.
5. Schleit J, Johnson S C, Bennett C F, et al. Molecular mechanisms underlying genotype-dependent responses to dietary restriction. Aging cell 2013; 12:1050-61.
6. Johnson S C, Yanos M E, Kayser E B, et al. mTOR inhibition alleviates mitochondrial disease in a mouse model of Leigh syndrome. Science 2013; 342:1524-8.
7. Johnson S C, Yanos M E, Bitto A, et al. Dose-dependent effects of mTOR inhibition on weight and mitochondrial disease in mice. Front Genet 2015; 6:247.
8. Kreis H, Oberbauer R, Campistol J M, et al. Long-term benefits with sirolimus-based therapy after early cyclosporine withdrawal. J Am Soc Nephrol 2004; 15:809-17.
9. Canaud G, Bienaime F, Tabarin F, et al Inhibition of the mTORC pathway in the antiphospholipid syndrome. N Engl J Med 2014; 371:303-12.
10. Canaud G, Bienaime F, Viau A, et al. AKT2 is essential to maintain podocyte viability and function during chronic kidney disease. Nat Med 2013; 19:1288-96.
11. Schneider C A, Rasband W S, Eliceiri K W. NIH Image to ImageJ: 25 years of image analysis. Nature methods 2012; 9:671-5.

1. A method for treating mitochondrial genetic diseases in a subject in need thereof comprising administrating to the subject a therapeutically effective amount of a PI3K inhibitor.

2. The method according to claim 1, wherein the PI3K inhibitor is a small molecule.

3. The method according to claim 1, wherein the PI3K inhibitor is BYL719 (Alpelisib).

4. The method according to claim 1, wherein the PI3K inhibitor is GDC-0032 (Taselisib).

5. The method according to claim 1, wherein the mitochondrial genetic disease is Leigh Syndrome.

6. The method according to claim 1, wherein the mitochondrial genetic disease is ataxia.

7. The method according to claim 1, wherein the mitochondrial genetic disease is cerebellar hypoplasia.

8. The method according to claim 1, wherein the mitochondrial genetic disease is kearns-sayre syndrome.

9. A method of screening a drug suitable for the treatment of mitochondrial genetic diseases comprising i) providing a test compound ii) determining the ability of said test compound to inhibit the activity of PI3K, and, based on results from the determining step, iii) identifying the test compound as a suitable drug.
